(19) United States
(12) Patent Application Publication
(10) Pub. No.: US 2021/0315728 A1
(43) Pub. Date: Oct. 14, 2021

(54) ADJUSTABLE DENTAL HINGE SYSTEM AND METHOD FOR MANDIBLE POSITIONING

(71) Applicant: Mark Abramson, Redwood City, CA (US)

(72) Inventor: Mark Abramson, Redwood City, CA (US)

(21) Appl. No.: 16/917,479

(22) Filed: Jun. 30, 2020

Related U.S. Application Data
(60) Provisional application No. 62/869,693, filed on Jul. 2, 2019.

(51) Int. Cl. A61F 5/56

(52) U.S. Cl. CPC A61F 5/566

(57) ABSTRACT

A dental device adapted to provide forward relative positioning of a mandible of a patient including: a mandibular tray formed to the patient's mandibular dentition including a mandibular attachment wire encased within an outer circumference of the mandibular tray; a maxillary tray formed to the patient's maxillary dentition, the maxillary tray including a maxillary attachment wire encased within an outer circumference of the maxillary tray; and a pair of hinge devices coupled to the mandibular tray via the mandibular attachment wire and coupled to the maxillary tray via the maxillary attachment wire, each hinge device including a housing having an inner channel and a telescoping member movable within the inner channel to change a length of the hinge device to enable forward mandibular advancement, the telescoping member including precise circumferential grooves.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)). All subject matter of the Related Applications and of any and all parent, grandparent, great-grandparent, etc. applications of the Related Applications is incorporated herein by reference to the extent such subject matter is not inconsistent herewith.

The present application is a non-provisional filing of, and claims benefit under 35 U.S.C. § 119(e) from, U.S. Provisional Patent Application Ser. No. 62/869,693, entitled “ADJUSTABLE DENTAL HINGE SYSTEM AND METHOD FOR MANDIBLE POSITIONING,” filed Jul. 2, 2019, which is hereby incorporated by reference in its entirety.

### BACKGROUND

Sleep apnea continues to be a common risk factor for high blood pressure, heart attack, and stroke. Continuous positive airway pressure (CPAP) or an automatic positive airway pressure (APAP) device forces a patient's airway open during sleep by a flow of pressurized air into the throat. A patient typically wears a plastic facial mask, which is connected by a flexible tube to a small bedside CPAP machine that generates the required air pressure to keep the patient's airways open during sleep. In addition, an APAP machine will automatically titrate the air pressure as needed to minimize apneas and hypopneas. Advanced models may warm or humidify the air and monitor the patient's breathing. Although CPAP and APAP therapy is effective in reducing apneas, many patients find the therapy extremely uncomfortable and fail to continue using their CPAP machines on a nightly basis, especially in the long term. Further, it is not clear that CPAP therapy reduces hypertension or cardiovascular events in patients who do not have daytime sleepiness; however, the lack of benefit may be partly due to noncompliance with therapy.

Alternatively, custom-fitted sleep disorder devices are available and designed to help patients sleep soundly and continuously, while reducing the effects of sleep apnea. Patients with sleep apnea and snoring issues are individuals with unique issues making a one treatment fits all type device impossible. Thus, additional options are needed for reducing obstruction in airway paths of patients to treat sleep apnea and snoring so that patients can choose an option that is most comfortable for them and therefore allows them to follow through with treatment on a more consistent basis, thereby improving treatment results.

### SUMMARY

One or more embodiments are directed to a dental device adapted to provide forward relative positioning of a mandible of a patient including: a mandibular tray formed to the patient's mandibular dentition including a mandibular attachment wire encased within an outer circumference of the mandibular tray; a maxillary tray formed to the patient's maxillary dentition, the maxillary tray including a maxillary attachment wire encased within an outer circumference of the maxillary tray; and a pair of hinge devices coupled to the mandibular tray via the mandibular attachment wire and coupled to the maxillary tray via the maxillary attachment wire, each hinge device including a housing having an inner channel and a telescoping member movable within the inner channel to change a length of the hinge device to enable forward mandibular advancement, the telescoping member including precise circumferential grooves on a rod portion. In one or more embodiments, each of the mandibular tray and the maxillary tray include a pair of metal posts at a forward location relative to the hinge device to enable a pair of elastic bands to releasably couple the mandibular tray to the maxillary tray. Each hinge device further includes a first attachment structure extending from the housing for pivotably connecting the housing to the mandibular attachment wire and a second attachment structure extending from the telescoping member for pivotably connecting the telescoping member to the maxillary attachment wire.

In one or more embodiments the pair of elastic bands form a triangle on each side of a patient's mouth by coupling to each of the pair of metal posts and to the first attachment structure.

In an embodiment, an adjustable screw stop is positionable within the inner channel, wherein the inner channel is threaded, to block the telescoping member at a minimum length of the device. In an embodiment, at least a portion of a length of the inner channel is threaded and the stop is a nylon-coated set screw with threads mateable with the threads of the inner channel so that the stop is advanceable along the threads of the inner channel to select the minimum length. In an embodiment, the telescoping member is cylindrical in cross-section and the housing is cylindrical in cross-section.

In an embodiment, a proximal end of the telescoping member extends from the housing and a distal end of the telescoping member is moveable within the inner channel and a cross-section of the distal end of the telescoping member is larger than a cross-section of the proximal end of the telescoping member. The inner channel narrows at a proximal end for a last 1.5 mm of the inner channel so as to block movement of the telescoping member when the distal end of the telescoping member, which has a 1.25 mm larger at a last portion, contacts the narrowed proximal end of the inner channel.

In an embodiment, the first attachment structure of each hinge device housing is a fin that extends downward toward the mandibular attachment wire relative to the housing when each hinge device is fixed in a mouth of a patient and the second attachment structure is a fin that extends upward toward the maxillary attachment wire relative to the telescoping member when the device is fixed in a mouth of a patient.

In one or more embodiments, the first attachment structure is produced independent of nickel by forming the housing with the via a metal injection mold process.

In one or more embodiments, the telescoping member and the housing are fabricated from one or more of a surgical grade metal and a thermoplastic resin.

In one or more embodiments, the maxillary tray includes a pair of nasal dilators, each nasal dilator including a button adjustably connected to the maxillary tray by one or more wires.

In one or more embodiments, the mandibular tray includes a pair of tongue lifters, each tongue lifter adjustably connected to the mandibular tray. Each tongue lifter coupled to the mandibular tray via a wire.

In one or more embodiments, the tongue lifters and the nasal dilators include thermoplastic material.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is perspective view of a dental device in accordance with one or more embodiments of the present disclosure.

FIG. 2 is perspective view of a dental device in accordance with one or more embodiments of the present disclosure.

FIG. 3 is perspective view of a dental device in accordance with one or more embodiments of the present disclosure.

FIG. 4 is a side view of hinge device for use with the dental device in accordance with one or more embodiments of the present disclosure.

FIG. 5 is a side view of hinge device for use with the dental device in accordance with one or more embodiments of the present disclosure.

FIG. 6 is a perspective view of hinge device for use with the dental device in accordance with one or more embodiments of the present disclosure.

FIG. 7 is a side view of hinge device for use with the dental device in accordance with one or more embodiments of the present disclosure.

FIG. 8 is a side view of hinge device for use with the dental device in accordance with one or more embodiments of the present disclosure.

FIG. 9 is a side view of hinge device for use with the dental device in accordance with one or more embodiments of the present disclosure.

FIG. 10 is a side view of hinge device for use with the dental device in accordance with one or more embodiments of the present disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

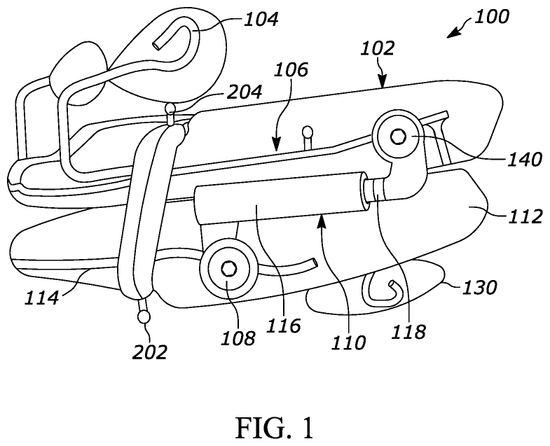

Referring to FIG. 1, one or more embodiments are directed to a dental device 100 adapted for sleep apnea including a maxillary tray 102, nasal dilators 104, maxillary attachment wire 106, first attachment area 108, hinge 110, mandibular tray 112, mandibular attachment wire 114, rod 118, and tongue lifters 130. Dental device 100 provides forward relative positioning of a mandible of a patient via mandibular tray 112 formed to the patient's mandibular dentition including a mandibular attachment wire 114 encased within an outer circumference of the mandibular tray and a maxillary tray 102 formed to the patient's maxillary dentition, the maxillary tray including a maxillary attachment wire 106 encased within an outer circumference of the maxillary tray. FIG. 1 is an embodiment using a hinge device in combination with other devices to treat severe sleep apnea, including congestion and airway blockage. Side hinges 110 provide 10 mm of possible advancement of a mandible and includes screws to enable 1-2 mm of retraction. Advantageously, the hinges hold a lower jaw forward during sleep while allowing a patient to open and close the mouth with some side to side movement. Thus, an aid to sleep apnea by opening an airway in the back of the throat and pulling a tongue forward. Unlike prior art hinge designs, which suffered from wobble and telescoping member 118, one or more embodiments the front attachment fin, attaching to the mandibular tray the center point of the first attachment fin 108 is no more than 2.5 mm from the bottom of hinge 110. Additionally, each of first attachment structure 108 and second attachment structure 140 attachment screws to enable elastic bands to optionally couple not only posts 202 and 204, but to the first attachment fin as well (shown in FIG. 3).

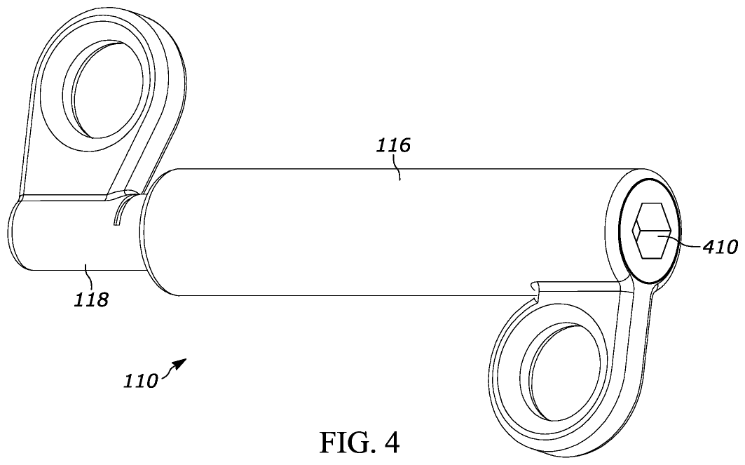

As shown in FIG. 1, hinge device 110 is included as a pair of hinge devices, shown in detail in FIGS. 4-10, coupled to the mandibular tray 112 via the mandibular attachment wire 114 and coupled to the maxillary tray 112 via the maxillary attachment wire 106, each hinge device 110 include a housing 116 having an inner channel and a telescoping member 118 movable within the inner channel to change a length of the hinge device 110 to enable forward mandibular advancement.

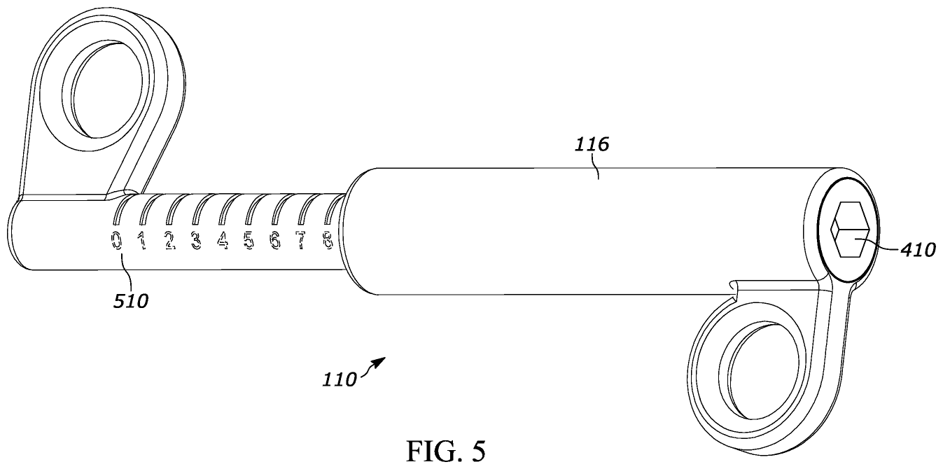

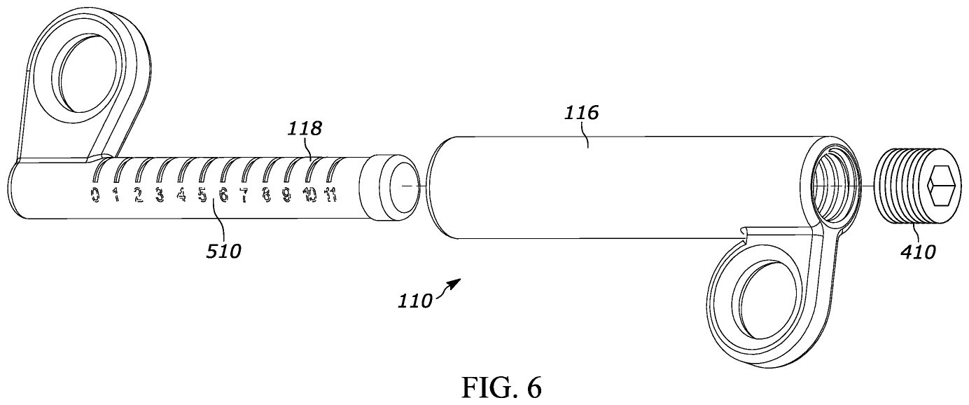

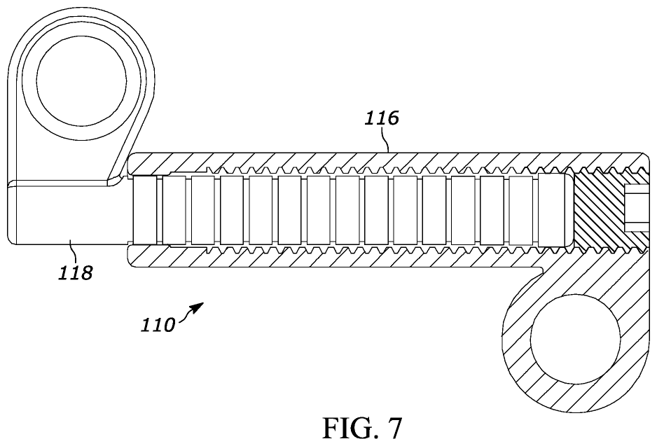

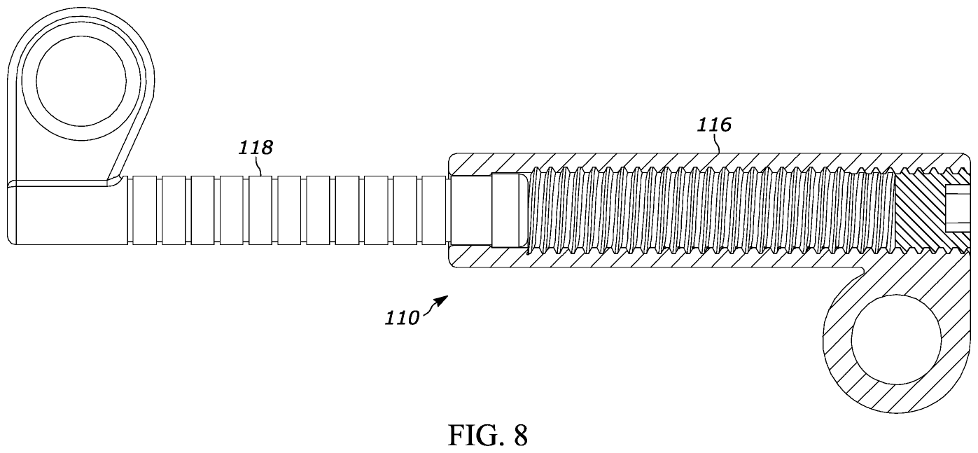

In one or more embodiments, the telescoping member 118 includes precise measurement indentations as shown in FIGS. 5-10, at, for example, millimeter distances apart. In one or more embodiments, the measurement indices include all or part of the entire circumference of the telescoping member rod. The measurement indices can be at each millimeter or other appropriate distance around the rod so that a patient can easily determine the amount of mandibular advancement caused by the telescoping action. FIGS. 5 and 6 illustrate the indices 510 for measurement at millimeter distances, shown in dotted lines for reference purposes. Referring to FIGS. 7 and 8, a representation of the hinge device 110 illustrates circumferential grooves to enable a patient to identify the number of millimeters of advancement of the mandible. FIG. 7 illustrates a fully inserted telescoping member 118. FIG. 8 illustrates a fully advanced mandible position for hinge device 110 showing telescoping member 118 fully extended. The combination of the circumferential grooves about at least part of a cross section of the telescoping member 118 that are equidistant and a curved entry to the housing of hinge 110 benefit a patient by allowing free movement of the telescoping member 118 within the housing without catching on an edge of the housing upon entry.

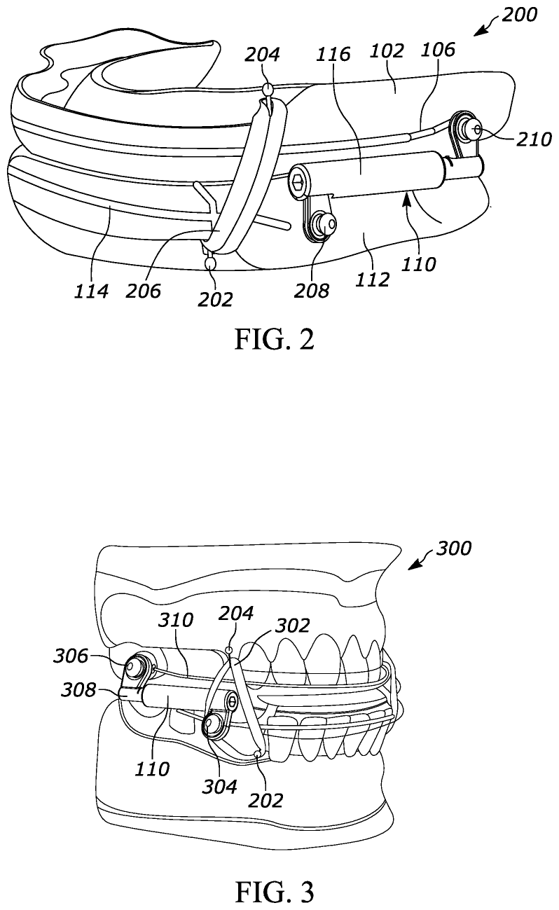

Referring to FIG. 2, in one or more embodiments, each of the mandibular tray and the maxillary tray include a pair of metal posts 202, 204 at a forward location on each side of the patient, relative to the hinge device 110 to enable a pair of elastic bands 206 to releasably couple the mandibular tray 112 to the maxillary tray 102. FIGS. 1 and 2 illustrate use of the two posts on a left side of a patient. Each elastic band can also form a triangular shape 302 as shown in FIG. 3 by including the first attachment structure with a screw 304 or stay within the structure functioning to hold the elastic band 206 to further prevent wobble or other unwanted movement of the hinge device 110.

In other embodiments, as shown in FIG. 2, each hinge device 110 can include attachment screws 208, 210 within one or more of the fin-like attachment structures to enable secure coupling to each of the maxillary 102 and mandibular 112 trays.

In one or more embodiments, each hinge device 110 can include a first attachment structure such as fin-like attachment structures 208 and 210 that pivotably connects the hinge 110 to the mandibular attachment wire 114, and a second attachment structure 210 extending from the telescoping member 118 for pivotably connecting the telescoping member 118 to the maxillary attachment wire 106.

Referring to FIG. 3, in one embodiment, dental device 100 is shown on a model 300 to illustrate that, in one embodiment, an appliance 310 can have elastic 302 form a triangle around the first attachment area 304 to reduce wobble of hinge 110. Also shown is telescoping member area 308 and second attachment area 306.

Referring to FIG. 4, each hinge device 110 can further include a stop screw 410 as an elastic stop band or metallic stop screw adjustably positionable to selectively limit movement of the telescoping member within an inner channel of the housing.

In one or more embodiments, the stop 410 is positionable within the inner channel to block the telescoping member 118 at a minimum length of the device. In an embodiment, at least a portion of a length of the inner channel is threaded and the stop is a nylon-coated set screw with threads mateable with the threads of the inner channel so that the stop is advanceable along the threads of the inner channel to select the minimum length. In an embodiment, the telescoping member 118 is cylindrical in cross-section and the hinge 110 is cylindrical in cross-section.

In an embodiment, a proximal end of the telescoping member 118 extends from the hinge 110 and a distal end of the telescoping member 118 is moveable within the inner channel and a cross-section of the distal end of the telescoping member 118 is larger than a cross-section of the proximal end of the telescoping member 118. The inner channel narrows at a proximal end of the inner channel (mandibular attachment end) so as to block movement of the telescoping member 118 when the distal end of the telescoping member 118 contacts the narrowed proximal end of the inner channel.

In an embodiment, referring to FIGS. 1-3, the first attachment structure of each hinge 110 is a fin-like structure holding an attachment screw-like member 208 304, as shown in FIGS. 2 and 3. In one or more embodiments, the first attachment structure 208,304 coupled to the hinge 110 includes a circular attachment member, such as a screw within the opening of the hinge 110 first attachment opening 208, 304, connected to the mandibular attachment wire 114 when each hinge 110 is fixed in a mouth of a patient.

In one or more embodiments, a second attachment structure 210, 306 is a fin that extends upward toward the maxillary attachment wire 106, 310 relative to the telescoping member 118 when the device is fixed in a mouth of a patient. In one or more embodiments, as shown in FIGS. 1-10, the first attachment structure 208, 304 is closer to the hinge 110 than the second attachment structure 210, 306 to prevent wobble and maintain stability within a patient's mouth. Importantly, in one or more embodiments, from the bottom of the hinge device to the center point of the first attachment structure 208, 304 is no more than 2.5 mm in distance from the edge of hinge 110.

To further reduce wobble, as shown in FIG. 2 and FIG. 3, elastic bands 206, 302 can be configured to connect the maxillary tray 102 to the mandibular tray 112 in a triangular pattern formed by the maxillary tray post 204, the mandibular tray post 202, and around the first attachment structure screw 304. The first attachment structure screw 304 can include an elastic member integrated with the attachment screw, or be part of a metal injection molding process or the like. As one of skill in the art will appreciate, the placement of the elastic bands 302 can also be positioned as part of the hinge 110 as appropriate to increase stability of the hinge 110. In one or more embodiments, as shown in FIG. 3, the pair of elastic bands 302 form a triangle on each side of a patient's mouth by coupling to each of the pair of metal posts and to the first attachment structure 304. As shown in FIG. 3, an elastic band 302 is shown coupling to a pair of posts and to a stay inserted into the center of first attachment structure 304.

In one or more embodiments, the first attachment structure 208, 304 is produced independent of nickel by forming the housing with the via a metal injection mold process.

In one or more embodiments, the telescoping member 118 and the housing 110 are fabricated from one or more of a surgical grade metal and a thermoplastic resin.

In an embodiment, a system adapted to limit relative positioning of a mandible and a maxilla of a patient comprises a pair of devices (hinge 110) connectable between the mandible and the maxilla and positioned on opposite sides of the dental arches of the mandible and the maxilla. Each of the devices include a hinge 110 including an inner channel and a telescoping member 118 movable within the inner channel to change a length of the device. Each device further comprises a first attachment structure 208, 304 extending from the housing of hinge 110 for pivotably connecting the hinge 110 to one of the mandible and the maxilla and a second attachment structure 210, 306 extending from the telescoping member 118 for pivotably connecting the telescoping member 118 to the maxilla. Each device further comprises a stop adjustably positionable to selectively limit movement of the telescoping member 118 within the inner channel.

Referring to FIGS. 4-10 a hinge device is shown with an adjustable, telescoping member is attachable to the maxillary and mandibular trays shown in FIGS. 1-3 in position relative to each other. The device of FIGS. 4-10 is attachable via attachment structures that can be pivotably connected to dental trays. Such mounting structures can vary, and can be fixedly attached to the dental trays or removably attached to the dental arches. For example, such mounting structures can include fixed orthodontic wires to braces or to removable maxillary and mandibular dental trays, preferably though not necessarily thermoplastic and selectively insertable or removable. In an embodiment, the hinge system can comprise two devices positioned on opposite sides of the maxillary and mandibular dental appliances.

Each of the hinge devices of FIGS. 4-10 are shown independent of the attachment screws shown in the void space of the first and second attachment structures (holes). As one of skill in the art will appreciate, different attachment members can be used to attach the hinge device to the maxillary and mandibular trays.

The device in FIGS. 4-10 includes a hinge 110 having an inner channel. FIG. 4 illustrates an embodiment of the hinge device in a closed position.

FIG. 5 illustrates an embodiment of the hinge device in an open position.

FIG. 6 illustrates an embodiment of the hinge device separate with a

The inner channel is shown in FIGS. 6-10. The hinge 110 is positioned anterior to a telescoping member 118 shown in FIGS. 5 and 6 with measurements for insertion shown in dashed numbers for reference purposes only. As shown, FIGS. 5 and 6 includes a housing 116 having a substantially cylindrical cross-sectional shape, although as described below, in other embodiments, the housing 116 can have some other cross-sectional shape. A fin-like first attachment structure extends from the hinge 110 and allows the hinge device 110 to be pivotably connectable to the mandibular dental tray 114 formed to the dentition of a patient. The first attachment structure 208, 304 can be a fin-like extension that can be integrally formed with the hinge 110 via metal injection molding or separately attached via soldering to the housing.

In one embodiment, the first attachment structure 208, 304 is no more than 2.5 mm to the center point of mandibular fin to the bottom of the hinge 110, and no more than 5 mm from the bottom edge of the housing of the hinge device 110 to the bottom of the front fin. As described above, with reference to FIG. 1-3, first attachment structure 208, 304 includes a substantially circular eyelet for attachment to the mandibular dental tray 112 via a connector wire 114 that allows the hinge 110 to pivot smoothly about a connector via rotation. Pivoting occurs with the relative movement of the mandible and maxilla both with posterior and anterior movements of the mandible relative to the maxilla that occur with shifts in the setting of the teeth, and also with the opening and closing of the mouth during eating, speaking, etc.

Referring to FIG. 6, in one or more embodiments, an inner channel of the housing 116 includes screw threads for screw 410 extending from the anterior end of the housing 116 along at least a portion of the inner channel toward the posterior end of the housing 116 into the telescoping measured structure. The posterior-most end of the housing 116 has a narrowed (i.e. necked) section at the beginning of hashes shown with numbers 510 shown in dashed line for illustration purposes only with an internal cross-section (e.g., an internal diameter, where the housing is a tube) that is narrower than the rest of the inner channel. The narrowed section is forward of a patient's mouth and acts as a blocking structure limiting motion of the telescoping member 118 within the inner channel, thereby defining a maximum length of the hinge device 110.

As shown, in FIG. 6, the telescoping member 118 is a telescoping member (rod) having a larger (e.g. flared) external diameter anterior end that is arranged in the tube. The telescoping member can move within the inner channel of the housing 116 in a posterior direction until contacting the narrowed section toward a front of a patient's mouth having a diameter smaller than the anterior end of the telescoping member 118. The narrowed section impedes further posterior movement of the telescoping member and prevents the telescoping member from slipping through the posterior end of the housing 116 and separating from the housing 116. In one embodiment, the telescoping member 118 is initially placed into the housing 116 from a forward position, extended through the housing 116 until the larger circumference of the back end of the telescoping member 118 meets resistance in the housing. The second attachment structure, i.e., maxillary attachment (fin) is then soldered or formed by injection molding with the telescoping member 118.

The hinge device telescoping member 118 with the second attachment structure 210, 306 becomes the posterior-most component in a patient's mouth and includes the telescoping member 118 that slides telescoping back and forth through the hinge 110. In an embodiment, the telescoping member 118 can be arranged within the housing 116 by inserting the telescoping member 118 into the anterior end of the inner channel of the housing 116 and urging the telescope member 118 through the posterior end of the inner channel, positioning the telescoping member 118 so that it extends out of the posterior end of the housing 116. A second attachment structure can then be coupled by soldering the telescoping member positioned within the inner channel. The second attachment structure extends from the telescoping member 118 and allows the hinge device 110 to be pivotably connectable to the maxillary dental tray as shown in FIGS. 1-3.

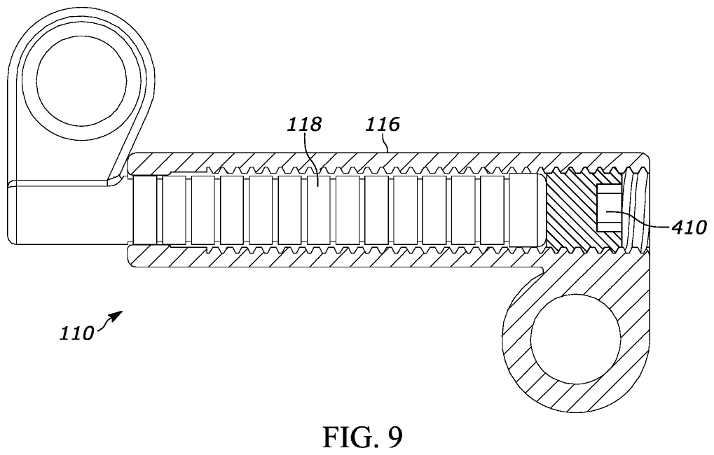

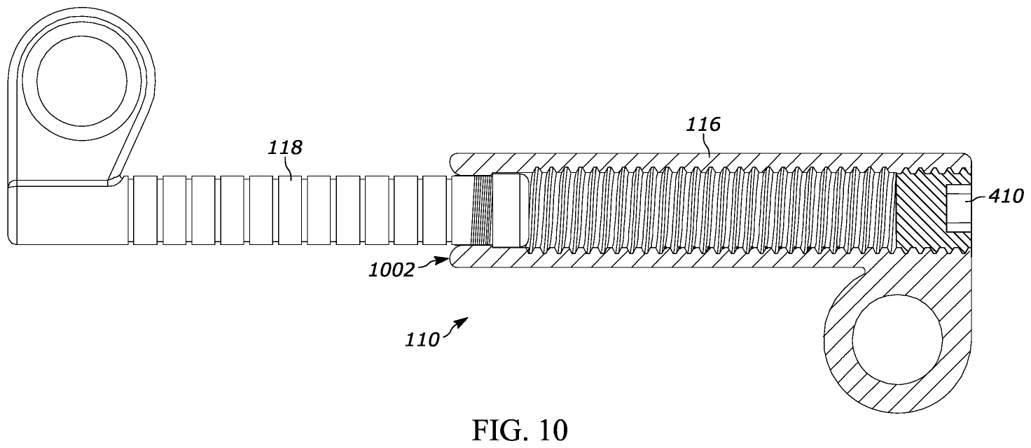

Referring to FIG. 7, hinge 110 is shown in cross section, showing telescoping member 118 fulling inserted into housing 116. FIG. 8 illustrates telescoping member 118 at a fully elongated position within hinge 110. FIG. 9 illustrates hinge 110 in cross section with telescoping member fulling inserted in housing 116 and with stop screw 410 inserted. FIG. 10 also illustrates hinge 110 with telescoping member 118 fully elongated outside housing 116, with stop screw 410 fulling inserted. Stop screw 410, which can be adjustably positionable to selectively limit movement of the telescoping member within the inner channel. In an embodiment, the stop screw 410 can comprise an adjustment screw threaded and coated with a liquid nylon, such a Nylok™ into the anterior end of the tube and advanced along the inner screw thread of the tube. In one embodiment, the stop screw 410 meets but cannot exceed a fully inserted telescoping member (rod). Thus, the rod meets but does incapable of pushing the stop screw from the housing. When the rod (telescoping member) is moved forward in the tube, the anterior-most end of the rod will meet the adjustment screw (FIG. 6), which creates a stop. In one or more embodiments, the adjustment screw is at least 3 mm in length.

In one embodiment, the lower mandibular tray 110 is prevented from moving in a posterior direction relative to the upper maxillary tray via the adjustment set screw 410 but allows forward or anterior movement of the mandibular tray because of the telescoping member 118 ability to slide in a posterior direction in the tube.

FIG. 10 illustrates a curved entry 1002 to the housing 116 to enable ease of sliding and to prevent the telescoping member 118 from catching on an edge of the housing 116 as it enters or retreats from the inner chamber of the hinge 110.

As shown in FIGS. 4, 5 and 6, the adjustment set crew 410 has an internal hex receptor for an Allen wrench to turn the screw. When turned to move posterior in the tube it changes the position of the stop making the hinge adjustable and the telescoping member 118 ability to move freely in the posterior direction makes it telescopic. In other embodiments, some other type of mating feature can be used for advancing and retreating the adjustment set screw.

The housing, telescoping member, attachment structures and stop screw can each include a metal, or some other suitable material, for example a thermoplastic resin such as polyetheretherketone (PEEK), and the like, or a combination of materials. In one embodiment, as mentioned above, the housing and the first attachment structure can be integrally manufactured or separately manufactured and subsequently attached. For example, in one embodiment, the first attachment structure 208, 304 and the housing 116 are manufactured using metal injection molding to advantageously limit the amount of nickel in the final hinge device caused by soldering. Further, the housing 116 and the first attachment structure 208, 304 can include the same or different materials, or combinations of materials. Further, in alternative embodiments, the telescoping member (rod) 118 and the second attachment structure 210, 306 can be integrally manufactured or separately manufactured and subsequently attached. Further, the telescoping member 118 and the second attachment structure 210, 306 can include the same or different materials, or combinations of materials. Selection of materials can be based on myriad different concerns, including whether the device is to be attached to fixed orthodontic wires or a removable, soft material.

In one or more embodiments, the telescoping member 118 can be the anterior-component connected with the maxillary dental arch and the housing 116 can be the posterior-component coupled with the mandibular dental tray 112. In other embodiments, the telescoping member 118 can be the anterior-component connected with the mandibular dental tray 112 and the housing 116 can be the posterior-component connected with the maxillary dental tray 102.

In alternative embodiments, some other technique(s) and/or structures can be used for positioning the telescoping member inside of the inner channel. For example, the narrowed end of the inner channel can be separately threaded onto the rest of the inner channel after a main length of a telescoping member is positioned through the narrowed end and a threaded anterior end of the telescoping member can be separately mated with the main length of telescoping member and advanced until the threaded anterior end is fully seated onto the main length of the telescoping member. Separating the telescoping member 118 into two threaded pieces and separating the inner channel into two pieces can simplify manufacturing and/or allow the attachment structure of the telescoping member to be integrally formed.

Embodiments described include but are not limited to, a dental device adapted to provide forward relative positioning of a mandible of a patient, the dental device including: a mandibular tray formed to the patient's mandibular dentition including a mandibular attachment wire encased within an outer circumference of the mandibular tray; a maxillary tray formed to the patient's maxillary dentition, the maxillary tray including a maxillary attachment wire encased within an outer circumference of the maxillary tray; and a pair of hinge devices coupled to the mandibular tray via the mandibular attachment wire and coupled to the maxillary tray via the maxillary attachment wire, each hinge device including a housing having an inner channel and a telescoping member movable within the inner channel to change a length of the hinge device to enable forward mandibular advancement, the telescoping member including precise circumferential grooves.

One or more embodiments include a dental device wherein each of the mandibular tray and the maxillary tray include a pair of metal posts at a forward location relative to the hinge device to enable a pair of elastic bands to releasably couple the mandibular tray to the maxillary tray.

One or more embodiments include a dental device wherein each hinge device further include a first attachment structure extending from the housing for pivotably connecting the housing to the mandibular attachment wire; and a second attachment structure extending from the telescoping member for pivotably connecting the telescoping member to the maxillary attachment wire.

One or more embodiments include a dental device wherein each hinge device includes an adjustable screw stop positionable within the inner channel, wherein the inner channel is threaded to block the telescoping member at a minimum length.

One or more embodiments include a dental device wherein the adjustable screw stop is at least three millimeters in length.

One or more embodiments include a dental device wherein at least a portion of a length of the inner channel is threaded and the adjustable screw stop is a nylon-coated set screw with threads mateable with the threads of the inner channel to identify a stop advanceable along the threads of the inner channel to select a minimum length.

One or more embodiments include a dental device wherein the housing has a curved edge where the telescoping member enters the housing to enable ease of movement of the telescoping member within the housing.

One or more embodiments include a dental device wherein a proximal end of the telescoping member extends from the housing and a distal end of the telescoping member is moveable within the inner channel; and a cross-section of the distal end of the telescoping member is larger than a cross-section of the proximal end of the telescoping member.

One or more embodiments include a dental device wherein the inner channel narrows at a proximal end for a last 1.5 mm of the inner channel to block movement of the telescoping member when a larger distal end of the telescoping member contacts the narrowed proximal end of the inner channel.

One or more embodiments include a dental device wherein the larger distal end of the telescoping member is 1.25 mm larger in diameter.

One or more embodiments include a dental device wherein the first attachment structure of each hinge device housing is a fin that extends downward toward the mandibular attachment wire relative to the housing when each hinge device is fixed in a mouth of a patient.

One or more embodiments include a dental device wherein the second attachment structure is a fin that extends upward toward the maxillary attachment wire relative to the telescoping member when the device is fixed in a mouth of a patient.

One or more embodiments include a dental device wherein the first attachment structure is produced independent of nickel by forming the housing with the via a metal injection mold process.

One or more embodiments include a dental device wherein the first attachment structure includes a structure in the center to enable an elastic band to couple the mandibular tray to the maxillary tray via a pair of metal posts at a forward location relative to the hinge device to enable a pair of elastic bands to releasably couple the mandibular tray to the maxillary tray via a triangular configuration.

One or more embodiments include a dental device wherein the telescoping member and the housing are fabricated from one or more of a surgical grade metal and a thermoplastic resin.

One or more embodiments include a dental device wherein the maxillary tray includes a pair of nasal dilators, each nasal dilator including a button adjustably connected to the maxillary tray by one or more wires.

One or more embodiments include a dental device wherein the mandibular tray includes a pair of tongue lifters, each tongue lifter adjustably connected to the mandibular tray via a wire.

One or more embodiments include a dental device wherein the pair of tongue lifters are produced with thermoplastic material.

One or more embodiments include a dental device wherein the pair of nasal dilators are produced with thermoplastic material.

One or more embodiments include a dental device wherein the first attachment structure extends no more than 5 millimeters from a proximate edge of the housing.

One or more embodiments include a dental device wherein the first attachment structure extends from the housing a shorter distance relative to the second attachment structure extension from the telescoping member.

One or more embodiments include a system adapted to advance a mandible of a patient, the system including a pair of hinge devices connectable between the mandible and the maxilla and positioned on opposite sides of a patient's mouth, each of the hinge devices including: a housing having an inner channel and a telescoping member movable within the inner channel to change a length of the respective hinge device; a first attachment structure extending from the housing for pivotably connecting the housing to a mandible, the first attachment structure extending no more than 5 millimeters from a proximate edge of the housing; a second attachment structure extending from the telescoping member for pivotably connecting the telescoping member toward a maxilla of a patient; and an adjustable set screw adjustably positionable within the housing to selectively limit movement of the telescoping member within the inner channel.

One or more embodiments include a system wherein the adjustable set screw is at least 3 millimeters in length;

One or more embodiments include a system wherein the housing has a curved edge where the telescoping member enters the housing.

One or more embodiments include a system wherein the telescoping member includes a plurality parallelly oriented circumferential grooves at millimeter spacing.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A dental device adapted to provide forward relative positioning of a mandible of a patient, the dental device comprising:
a mandibular tray formed to the patient's mandibular dentition including a mandibular attachment wire encased within an outer circumference of the mandibular tray;
a maxillary tray formed to the patient's maxillary dentition, the maxillary tray including a maxillary attachment wire encased within an outer circumference of the maxillary tray; and
a pair of hinge devices coupled to the mandibular tray via the mandibular attachment wire and coupled to the maxillary tray via the maxillary attachment wire, each hinge device including a housing having an inner channel and a telescoping member movable within the inner channel to change a length of the hinge device to enable forward mandibular advancement, the telescoping member including precise circumferential grooves.

2. The dental device of claim 1 wherein each of the mandibular tray and the maxillary tray include a pair of metal posts at a forward location relative to the hinge device to enable a pair of elastic bands to releasably couple the mandibular tray to the maxillary tray.

3. The dental device of claim 1 wherein each hinge device further comprises:
a first attachment structure extending from the housing for pivotably connecting the housing to the mandibular attachment wire; and
a second attachment structure extending from the telescoping member for pivotably connecting the telescoping member to the maxillary attachment wire.

4. The dental device of claim 1, wherein each hinge device includes an adjustable screw stop positionable within the inner channel, wherein the inner channel is threaded to block the telescoping member at a minimum length.

5. The dental device of claim 4, wherein the adjustable screw stop is at least 3 millimeters in length.

6. The dental device of claim 1 wherein at least a portion of a length of the inner channel is threaded and the adjustable screw stop is a nylon-coated set screw with threads mateable with the threads of the inner channel to identify a stop advanceable along the threads of the inner channel to select a minimum length.

7. The dental device of claim 1 wherein the housing has a curved edge where the telescoping member enters the housing to enable ease of movement of the telescoping member within the housing.

8. The dental device of claim 1 wherein:
a proximal end of the telescoping member extends from the housing and a distal end of the telescoping member is moveable within the inner channel; and
a cross-section of the distal end of the telescoping member is larger than a cross-section of the proximal end of the telescoping member.

9. The dental device of claim 1 wherein:
the inner channel narrows at a proximal end for a last 1.5 mm of the inner channel to block movement of the telescoping member when a larger distal end of the telescoping member contacts the narrowed proximal end of the inner channel.

10. The dental device of claim 9 wherein the larger distal end of the telescoping member is 1.25 mm larger in diameter.

11. The dental device of claim 3 wherein the first attachment structure of each hinge device housing is a fin that extends downward toward the mandibular attachment wire relative to the housing when each hinge device is fixed in a mouth of a patient.

12. The dental device of claim 3 wherein the second attachment structure is a fin that extends upward toward the maxillary attachment wire relative to the telescoping member when the device is fixed in a mouth of a patient.

13. The dental device of claim 3 wherein the first attachment structure is produced independent of nickel by forming the housing with the via a metal injection molding process.

14. The dental device of claim 3 wherein the first attachment structure includes a structure in the center to enable an elastic band to couple the mandibular tray to the maxillary tray via a pair of metal posts at a forward location relative to the hinge device to enable a pair of elastic bands to releasably couple the mandibular tray to the maxillary tray via a triangular configuration.

15. The dental device of claim 1 wherein the telescoping member and the housing are fabricated from one or more of a surgical grade metal and a thermoplastic resin.

16. The dental device of claim 1, wherein the maxillary tray includes a pair of nasal dilators produced with thermoplastic material, each nasal dilator including a button adjustably connected to the maxillary tray by one or more wires.

17. The dental device of claim 1, wherein the mandibular tray includes a pair of tongue lifters produced with thermoplastic material, each tongue lifter adjustably connected to the mandibular tray via a wire.

18. The dental device of claim 3 wherein the first attachment structure extends no more than five millimeters from a proximate edge of the housing to a lower edge of the first attachment structure.

19. A system adapted to advance a mandible of a patient, the system comprising:
a pair of hinge devices connectable between a mandibular tray and a maxillary tray and positioned on opposite sides of a patient's mouth, each of the hinge devices including:
a housing having an inner channel and a telescoping member movable within the inner channel to change a length of the hinge device, the housing including a curved edge where the telescoping member enters the housing;
a first attachment structure extending from the housing for pivotably connecting the housing to a mandible, the first attachment structure extending no more than 5 millimeters from a proximate edge of the housing;
a second attachment structure extending from the telescoping member for pivotably connecting the telescoping member toward a maxilla of a patient;
an adjustable set screw of three millimeters in length, adjustably positionable within the housing to selectively limit movement of the telescoping member within the inner channel;
at least two pairs of posts, each pair coupled on either side of a maxillary tray and a mandibular tray to enable a pair of elastic bands to provide stability to each hinge device;
a pair of tongue lifters disposed on the mandibular tray, each tongue lifter on either side of the mandibular tray and adjustably connected to the mandibular tray via a wire; and
a pair of nasal dilators coupled to the maxillary tray via a wire.

20. The system of claim 19 wherein the telescoping member includes a plurality parallelly oriented circumferential grooves at millimeter spacing.
